(19) United States
(12) Patent Application Publication
(10) Pub. No.: US 2012/0023785 A1
(43) Pub. Date: Feb. 2, 2012

(54) Wireless remote controlled massaging footwear system

(75) Inventors: Nathaniel Barnes, Berkeley, CA (US); Robert N. Ross, Gardner, MA (US); John W. C. Moehring, Pollock Pines, CA (US)

(73) Assignee: Nathaniel K. Barnes, Berkeley, CA (US)

(21) Appl. No.: 13/134,305

(22) Filed: Jun. 4, 2011

Related U.S. Application Data
(60) Provisional application No. 61/396,941, filed on Jun. 5, 2010.

(51) Int. Cl. A61F 5/14

(52) U.S. Cl. 36/141

(57) ABSTRACT

A new wireless remote controlled massaging footwear system, adaptable to typical footwear for individuals to wear and massage their feet during their normal daily activity. Massaging functions including multiple modes of operation and sequencing of massaging components is controlled using a wireless remote control device by the user or by a caregiver. The new system comprises massaging devices including six or more vibration motors and one or more various types of heating elements per foot that are structurally supported in enclosures. Enclosed massaging devices can be repositioned in the sole of the footwear to massage particular areas of the foot. The new system includes accessories such as a lanyard to wear the remote control around the user's neck and a carry case for protecting and transporting the system. The carry case is particularly suitable for a hospice care provider.

### CROSS-REFERENCE TO RELATED APPLICATIONS

This original nonprovisional application, is a continuation of provisional application No. 61/396,941 filed Jun. 5, 2010 by Nathaniel Barnes et al.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The new invention pertains to wireless remote controlled massaging footwear, in particular footwear that is controlled by wireless remote to massage the users feet while the user is walking or running.

Wireless remote controls are commonly used to operate many different types of devices. There are several common methods for wireless remote control operation, including; radio frequency (RF) and infrared (IR) communication, other newer wireless systems may also incorporate the use of cell phones and personal wireless electronic devices to remotely activate controls on other devices.

Foot massagers are well known devices. The most popular foot massaging devices include a stationary base having a defined area for the users' feet to rest against. The traditional foot massaging device is normally a large base unit plugged into an electrical wall outlet to power the massaging mechanism and heaters positioned in the foot massaging base. The user of these common foot massagers would sit in a chair and would need to take their shoes off to rest both their feet onto the single unit device in order to benefit from its massaging sensation. These typical devices are fairly large and not conducive for a care provider to bring along to visit a patient such as in hospice care. The user is confined to the chair and location of the massager.

Footwear is any type of manufactured article placed onto the feet to protect the feet from environmental conditions and injury while walking or running. Specific footwear is commonly made for particular purposes, conditions and environments. General categories for footwear include; Socks, Slippers, Sandals, Shoes, Sneakers and Boots.

Most people suffer from foot pain at some point during their lifetime. Foot pains are caused for various reasons, some examples are; injury, fatigue, medical conditions, including side effects from medications. Older people in particular tend to suffer from foot pains. Traditional foot massagers greatly help to stimulate and relax their feet.

A new and improved massaging footwear system would include built in and concealed wireless remote controlled massaging and heating components into ordinary footwear so that the user or a care giver can turn on the massager and heater when needed during the normal daily routine activities including sitting, standing, walking and running.

Massaging shoes are known in the art, U.S. Pat. No. 7,347,831 to Chiu teaches a ventilated shoe having an exposed first switch, and an exposed second switch mounted on the edge of a circuit board with the first and second switches provided on its edge. U.S. Pat. Nos. 5,913,838 and 5,836,899 to Reilly teaches electrical powered vibration footwear and insoles. U.S. Pat. No. 5,592,759 to Cox teaches the use of vibration motors in footwear. These system however, do not teach the use or the benefit of wireless remote control operation. Their design requires that the user needs to bend down to activate a switch on the shoe to functionally operate the heating element on the shoe and separately needs to bend down to activate a switch to functionally operate the vibration element. Chiu does not teach the wireless remote control to activate the shoe system.

### BRIEF SUMMARY OF THE INVENTION

Therefore, it is an object of this invention to provide a new and novel wireless remote controlled massaging footwear system, so that the user can change the footwear's massaging and heating operation settings while the user is sitting, walking or running. The system can further be controlled remotely by a caregiver to another person wearing the footwear, wherein said wireless remote controlled footwear system comprises; a hand held wireless remote control device, customized footwear, battery power source, a battery charger, optional lanyard and belt clip for said hand held wireless remote control and a carry case for said system. Said customized footwear further comprises a receiver for the remote control device, custom soles, enclosed electrical circuits, massaging components including; enclosed vibration motors and enclosed heating elements and other new technology devices as they come available such as; (Transcutaneous electrical nerve stimulation TENS and electrical muscle stimulation EMS devices or combination TENS/EMS devices).

It is also an object of the footwear system to provide structural support for said enclosed electrical circuits, enclosed vibration motors and heating elements and other massaging devices to withstand the weight of the user without damage to components. It is further an object of said enclosed heating elements to comprise electrical wire heaters or light energy emitting components having wavelengths in the visible or infrared, wherein said visible wavelength light energy emitting components comprise a supported array of miniature incandescent light bulbs, and infrared light energy emitting components comprise a supported array of light emitting diodes (LED's).

Said sole in said customized footwear may further comprise means to reposition said enclosed vibration motors and enclosed heating elements into desired locations within said sole for relief of specific foot aches.

### BRIEF DESCRIPTION OF SEVERAL VIEWS AND DRAWINGS

FIG. 1 is a perspective view of one embodiment of the system.

FIG. 2 is a cross sectional view of a shoe in FIG. 1.

FIG. 3 is an exploded assembly of one embodiment of a shoe and components.

FIG. 4 is an exploded assembly of one embodiment of the electrical circuit board and enclosure.

FIG. 5 is a cross sectional view of on embodiment of a vibration motor enclosure assembly.

FIG. 6 is a perspective view of one embodiment of a wireless remote control device.

FIG. 7 is a perspective view of one embodiment of a heating element enclosure assembly.

FIG. 8 is an exploded assembly of an alternate light energy emitting heating element and supportive enclosure.

FIG. 9 is a schematic circuit diagram for one embodiment of the remote control device.

FIG. 10 is a schematic circuit diagram for one embodiment of the receiver and electronic circuit to power massaging components.

### DETAILED DESCRIPTION OF THE DESIGN

The new wireless remote controlled massaging footwear system is intended to be integrated into any style footwear, however the detailed description of the design here will be specific to integrating the new system into an athletic style shoe as was actually built and tested. Alternate variations of components and features may be described throughout the description.

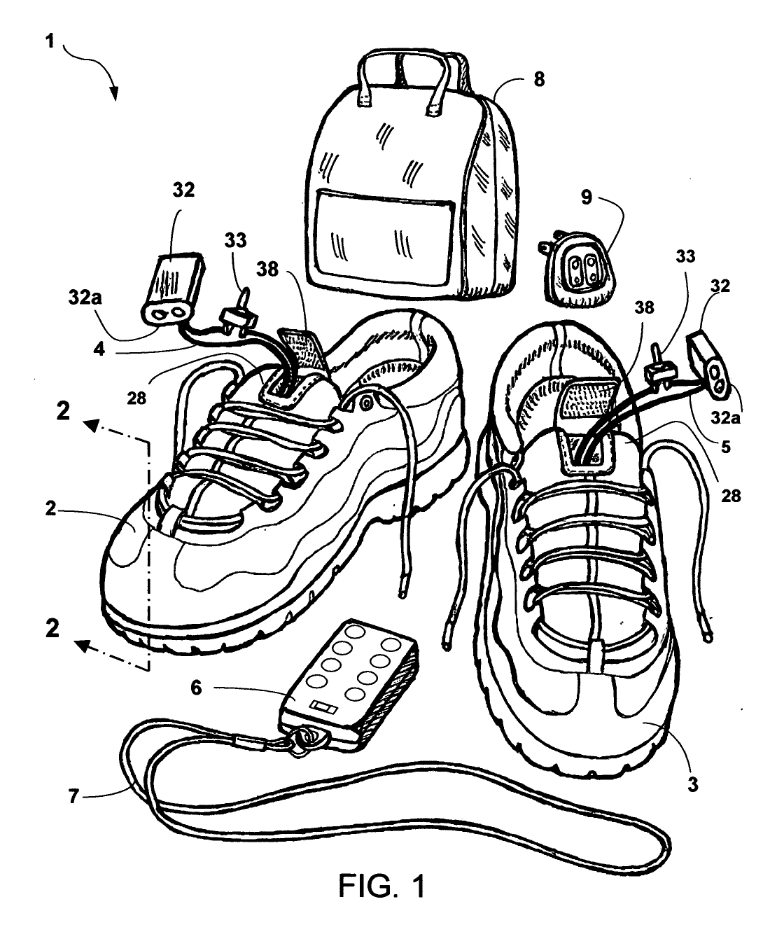

FIG. 1, describes one embodiment of the wireless remote controlled massaging footwear system 1 whose main components include; custom right and left shoes 2 and 3, having self contained battery powered electrical circuits 4 and 5 (partially shown), a wireless remote control 6 to operate electrical circuits 4 and 5 wherein battery 32, battery connector 32a and main switch 33 are concealed in the shoes tongue 28 and covered by flap 38 in both shoes 2 and 3. System accessories include a lanyard 7 for the remote control, belt clip (not shown), arm strap (Not shown), a carry case 8 for all accessories and a battery charging system 9.

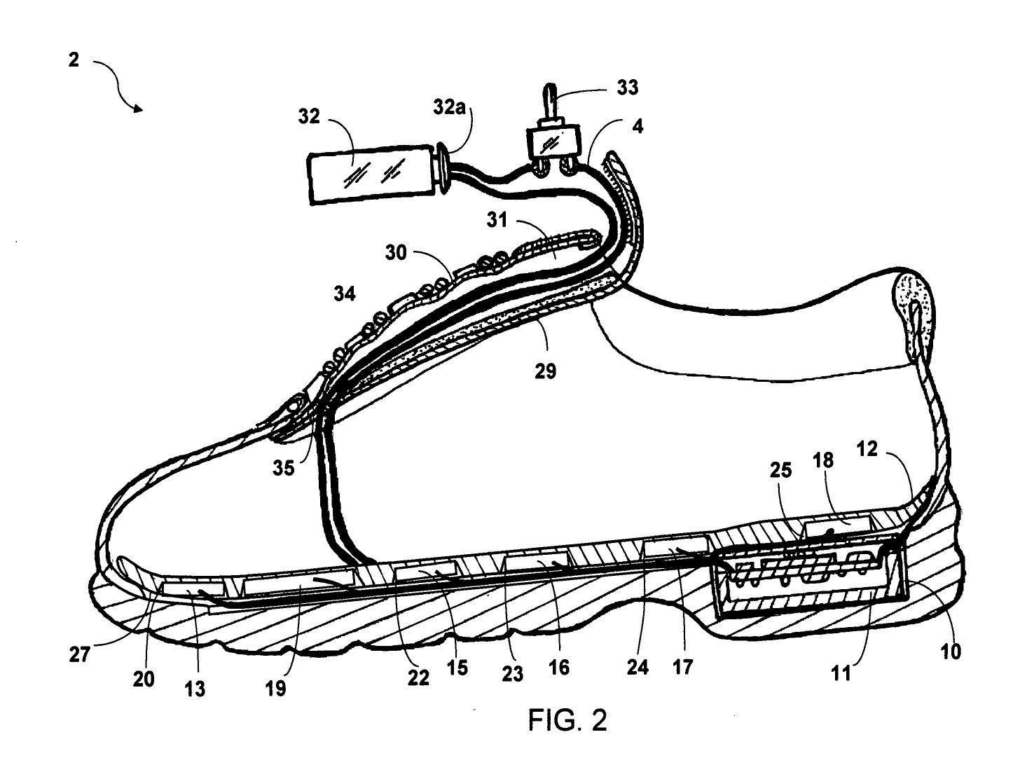

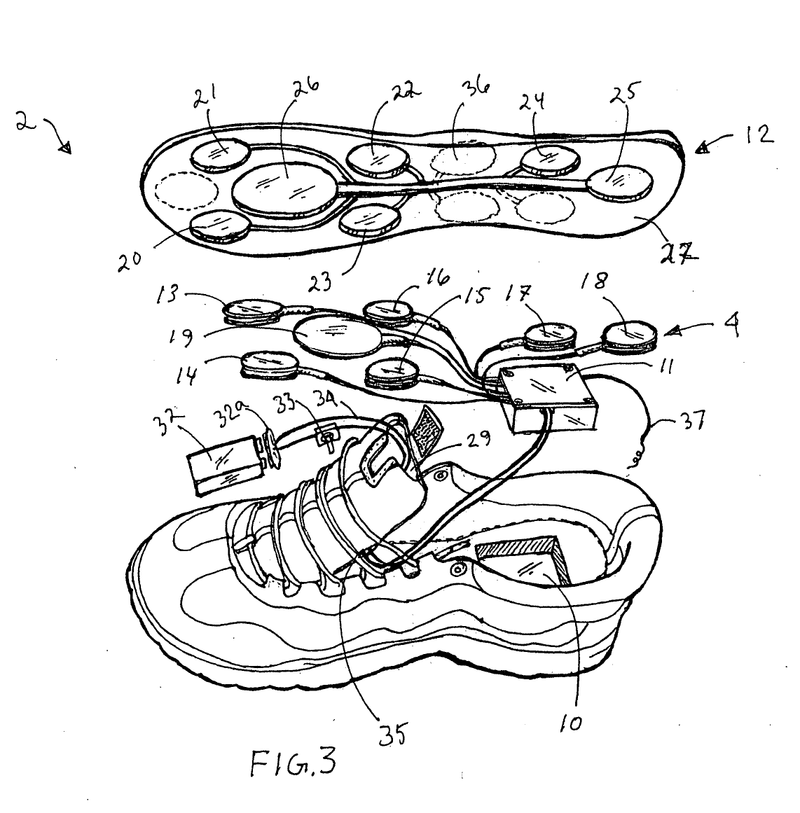

FIG. 2 describes the cross section of the right shoe 2 and the location and description of internal components. FIG. 3 describes an exploded view of the same components that are located within the shoe. Both right and left shoes of the system have essentially the same modifications and components. The modifications made to the traditional shoe are; a cavity 10 located in the heel portion of the sole of the shoe, where cavity 10 is sized for a snug fit to the electric circuit enclosure 11. A custom insole 12 is used to locate and position the enclosed vibration motors 13-18 and heating element 19. Custom insole 12 has recessed features 20-26 from the bottom surface 27 of the modified insole 12 to accommodate the enclosed vibration motors 13-18 and enclosed heating element 19. Extra recess cutouts 36 can also be added so that repositioning of massaging components can be accomplished as desired. The recess cutouts 36 may fully penetrate the insole else a thin protective layer will remain between the users foot and the massaging components. A tongue 28 on the custom shoe 2 having a tongue inner wall 29 and a tongue outer wall 30 thus forming an interior tongue space 31.

Interior tongue space 31 has adequate space to position some of the electrical wiring components including; a battery power supply 32, battery connector 32a, main switch 33 and a portion of the power supply wiring 34. The preferred battery power supply is a compact rechargeable lithium or metal hydride, low voltage, direct current, battery or battery pack. The prototype model was built using a rechargeable lithium, 9 volt, direct current, 200 milliamp hour, battery. The configuration of the main switch is at a minimum an on-off switch or preferably a three way switch having a charge position for recharging the battery installed within the shoe. The functional prototype model uses a simple on/off toggle switch. The switch 33, battery 32, battery connector 32a and a portion of the power supply wiring 34 would be positioned inside the interior tongue space 31. The power supply wiring 34 is inserted into the interior tongue space 31 through an opening in tongue seam 35. Seam 35 is then glued or sewn after insertion of power supply wiring 34. The exposed power supply wire 34 positioned inside the shoe would be covered by an adhesive backed fabric (not shown) so user's foot does not catch on wires and wires would then tuck underneath the custom insole 12 until they enter the electrical circuit enclosure 11. Antenna wire 37 also tucks under the custom sole 12.

Alternately if the footwear does not have a suitable tongue for creating the interior tongue space 31, the battery 32, switch 33 and power supply wiring 34 could be placed within electronic enclosure 11 positioned within cavity 10 in the sole of the shoe, else an independent enclosure can be employed, located within a separate cavity in the sole of the shoe, (not shown).

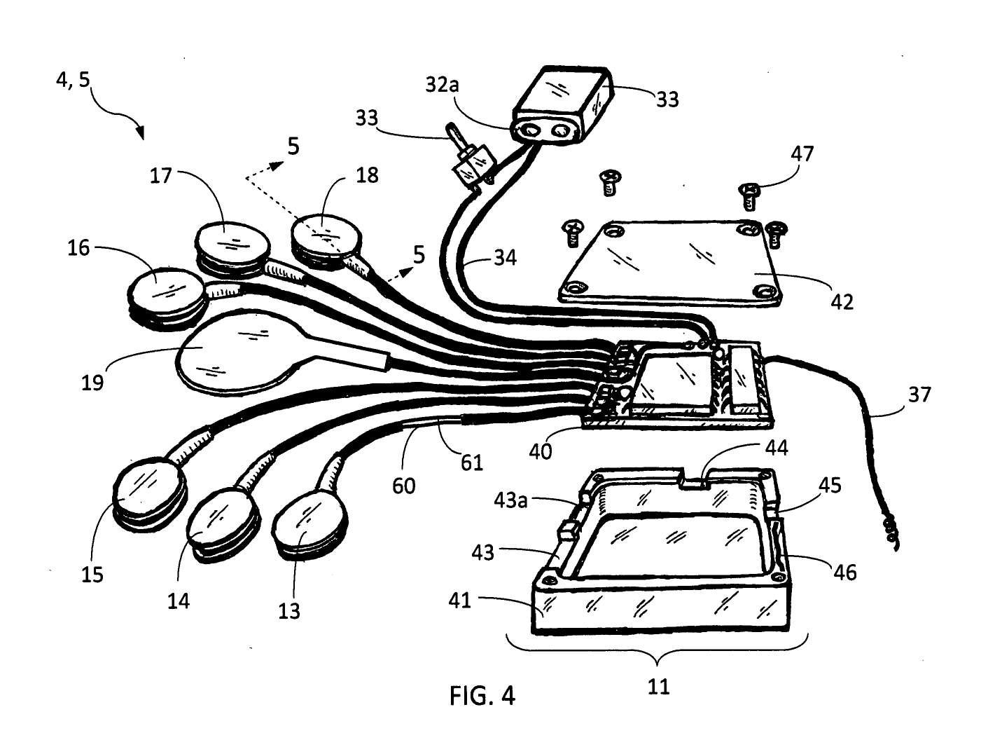

Referring to FIG. 4, the electric circuit enclosure 11 of the electrical circuits 4 and 5 is intended to provide structural support to the otherwise non structural electrical circuit board 40. Electrical circuit enclosure 11 also creates sealed protection to the electrical circuit board 40 from damage due to moisture or liquids that could short out electronics if footwear becomes wet. The electric circuit board 40 is comprised of electronic components for receiving the wireless signal from the hand held wireless remote control 6 to generate multiple output signals for independent, simultaneous or sequential operation of enclosed vibration motors 13-18 and heater 19. Production circuit boards can be mass produced integrated circuit boards and or flexible circuits, having thinner profiles to be more readily concealed within thinner heeled shoes. The electric enclosure base 41 is made from aluminum for strength and to dissipate any heat generated by the circuit board. The interior walls of the electric enclosure base 41 and inside surface of cover 42 are lined with a non conducting protective material such as adhesive backed film of polyamide, or other non conducting materials to prevent potential electrical shorting from occurring between circuit board components. To seal the enclosure, the circuit board 40 is positioned inside the electric board enclosure base 41, wires from the circuit board to the vibration motors and heaters are positioned through a first notch 43 and 43a in the base enclosure, the power supply wires fit through a second notch 44 and the antenna wire 37 fits through a third notch 45. Three notches exist in this embodiment since the wires are positioned on three sides of the circuit board, other embodiments may only require one or two notches, based on wire positioning. An RN or other non conducting sealant 46 is applied to the top edge of the electric circuit board base and to seal around the wires and to fill notches. The electric circuit board cover 42 is secured to the electric enclosure base using multiple screws 47. Cover 42 is made from 1.5 mm thick sheet metal for strength. Physical dimensions of the circuit board enclosure 11 on model built was 46 mm×46 mm×12 mm. Circuit board 40 dimensions were 38 mm×38 mm×10 mm.

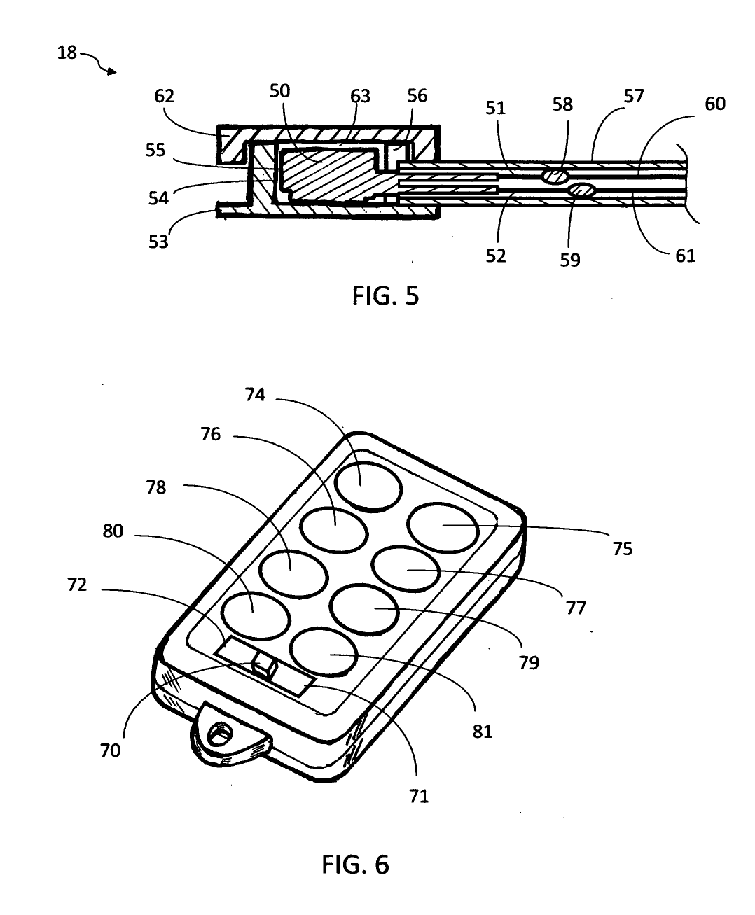

Referring to FIG. 5, the vibration motor enclosure 13 is described in a cross section view. The vibration motor 50 is delicate and needs to be properly supported to prevent damage. Vibration motors used are the same used in cell phones; operating voltage is 3.0-3.5 volts DC. The vibration motor wire leads 51 and 52 are thin and also need to be properly supported. The vibration motor enclosure 13 supports the vibration motor 50 by vibration motor enclosure bottom 53 having an internal counter bore 54 slightly larger then the vibration motor outside diameter 55, and a slot 56 positioned to create a snug fit to strain relief 57. Strain relief 57 is pushed against the vibration motor outside diameter and extends beyond the solder joint 58 and 59 connecting vibration motor leads 51 and 52 to circuit board connection leads 60 and 61. Strain relief 57 is bonded to wire leads and to the vibration motor bottom 53 using a flexible adhesive such as RTV. The vibration motor enclosure top 62 is secured to the vibration motor enclosure bottom to fully protect the vibration motor 50. A small space 63 remains between the vibration motor 50 and the vibration motor enclosure top 62 so that motor cannot stall due to the user's weight. The vibration motor enclosure bottom is made from aluminum for strength and to dissipate any heat generated away from the vibration motor 50. The outer envelope dimension of the vibration motor enclosure is less than 30 mm diameter. Due to the compact size and secure construction of the vibration motor enclosure 13 and the strength and flexibility of the circuit board connection leads 60 and 61, each vibration motor enclosure 13-18 can be independently positioned to a desired position for generating vibrations onto portions of the foot that are sore. To further assist with desired location for introducing massaging and heating, the wireless remote control 6 has built in controls to independently adjust the motor operation as the user desires.

FIG. 6 describes the functional features of one embodiment of the wireless remote control 6 as built and tested. The preferred wireless remote control uses a radio frequency (RF) signal to activate the massaging devices built into the electrical systems 4 and 5 in shoes 2 and 3 as previously described. The wireless remote control 6 will independently operate both shoe 2 and 3 simultaneously. A switch 70 turns the remote control operation to either shoe 2 in position 71 or to shoe 3 in position 72. Once the main switch 33 in the shoe is initially turned on, and the wireless remote control switch 70 is placed in position 71 it can be used to operate the messaging devices in shoe 2, then by pressing button 74 once this will turn on corresponding vibration motor V1 in shoe 2 or vibration motor enclosure 13 shown in FIG. 3. If button 74 is pressed again the vibration motor enclosure 13 in shoe 2 will turn off. (Additional signals can also be added to adjust the level of vibration by pushing the button once for low vibration, twice for medium vibration, three times for high vibration and a fourth time to turn off vibration). While vibration motor 13 is turned on using button 74 it will remain on while the remote control switch 70 is slid to position 72 to operate shoe 3, Now button 74 when pressed will start the corresponding vibration motor enclosure located in shoe 3. Both vibration motors are operating in shoe 2 and shoe 3. In order to turn off the vibration motor in either shoe, the switch 70 would be positioned to the desired shoe, and the corresponding button would turn the corresponding massaging device off or on and any other programmed feature in the button control. The same applies to the operation of the other buttons on the remote control 6 button 74-79 control the vibration motors 13-18 in shoe 2 and corresponding shoe 3. Button 80 controls the heater 19 in shoe 2 and corresponding shoe 3 in the same manner as was done for the vibration motors. Pressing button 80 will turn on, off or can also change heating levels. Button 81 generates a sequencing of selected vibration motors in programmed time intervals of operation, when button 81 is pressed vibration motor 13 will operate for two to four seconds then vibration motor enclosure 14 will operate for 2-4 second interval then vibration motor enclosure 15 will operate for 2-4 seconds and then vibration motor enclosure 16 will operate for 2-4 seconds. The sequencing process will continue until button 81 is pressed a second time to turn of the sequencing feature. Both shoes can operate in this manner by using switch 70. Other functional embodiments of the remote control 6 will consist of touch screen displays, the use of commercial cell phones to be programmed to operate the electric circuits, Infrared signals, and other technologies that are typically used for wireless communications.

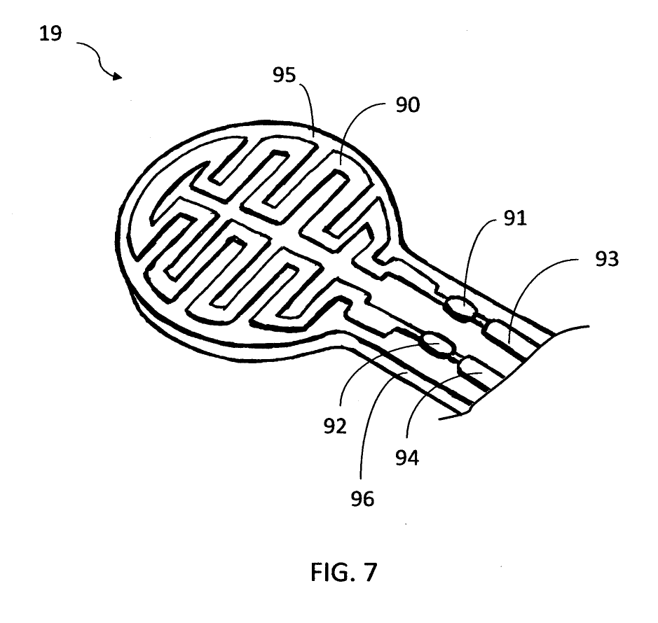

FIG. 7 describes a preferred embodiment for the heating element. Heating element enclosure 19 is a very thin simple thermal plastic coated electrical circuit that dissipates controlled conductive heat in temperature ranges less than 60 degree C. A metal resistance path 90 is etched form a thin metal sheet, shaped to have two solder points 91 and 92 creating a loop to connect directly into the electrical circuit. The positive circuit wire 93 is soldered to one of the ends 91 as shown and the negative circuit wire 94 is soldered to the other end 92 as shown to create electric current through the metal resistance path 90. A protective film 95 preferably adhesive backed polyamide plastic is used to coat the outer surfaces of the circuit including the metal resistance path 90 and solder joints 91 and 92 and portions of the circuit wires 93 and 94. An additional strain relief 96 made from shrink tubing or elastomer sheet can be added to further protect the solder joints 91 and 92 from damage in use due to flexing under foot. The resultant heating element enclosure 19 is very flexible and thin <0.02 inches in the area of the metal resistance path 90 and maximum thickness near solder joints 91 and 92 and strain relief less than 0.06 inch. There are endless shapes, sizes and configurations adaptable to this style of heating element. This heating element can also be integrated into a flexible circuit board encompassing all the electronics for the shoe. Also due to its thin profile the heating element may be positioned onto the top and or sides of the foot.

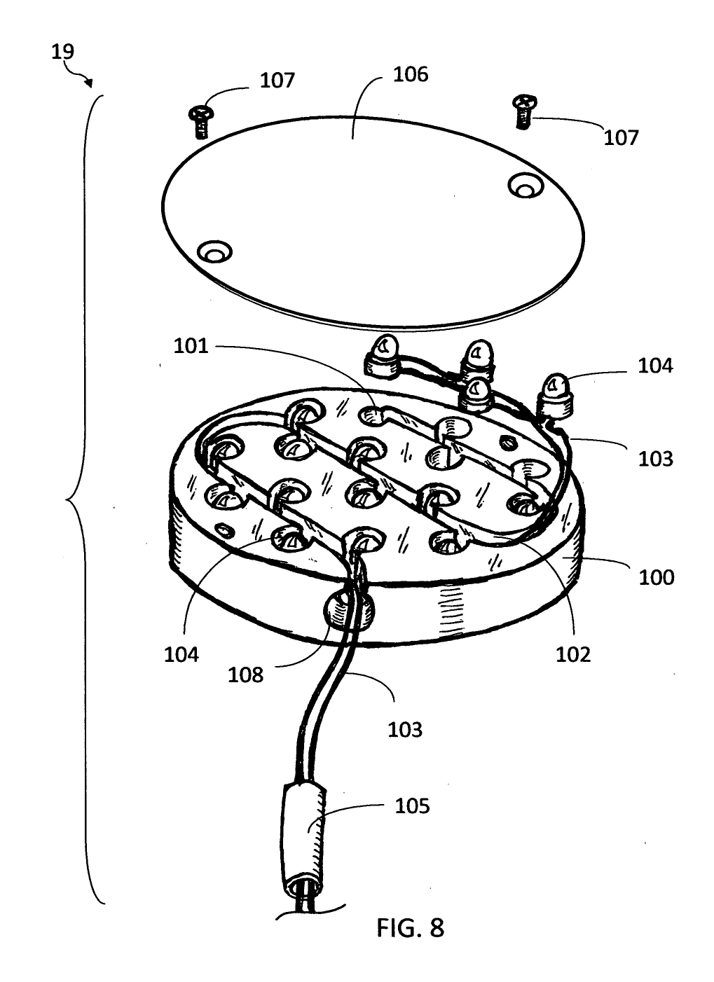

An alternate heating element enclosure 19 is shown in FIG. 8. Light energy emitted in different wavelengths provides muscle relaxation and stimulation at different depths below the skin's surface. Alternate heat energy can be accomplished using illumination via miniature incandescent light bulbs or LED's. Infrared light having wavelengths in the 940 nanometer range can stimulate muscle tissue beneath the skin surface to depths up to 1 inch. Light energy emitting components are rather delicate and need to be protected for use in the footwear. FIG. 8 describes one embodiment for a light emitting heating element enclosure. The component support structure 100 is comprised of an array of holes 101, each sized to support a light energy component 104. A pattern of slots 102 connects the holes together. Light energy component wire leads 103 lay within the pattern of slots 102 to exit the support structure 100 in a common strain relief 105 that fits into hole 108. A cover 106 is added and secured using screws 107 to protect and secure the light components into the assembly. Cover 106 can be either opaque to absorb the light energy and to act as a direct heating element to the foot or cover 106 may be transparent to transmit the light energy directly to the foot. Although the preferred assembly is sealed using RTV or equivalent sealant on all joints, the cover may also have through holes positioned over the output of the light energy component for more direct light exposure to the foot and reducing light energy loss.

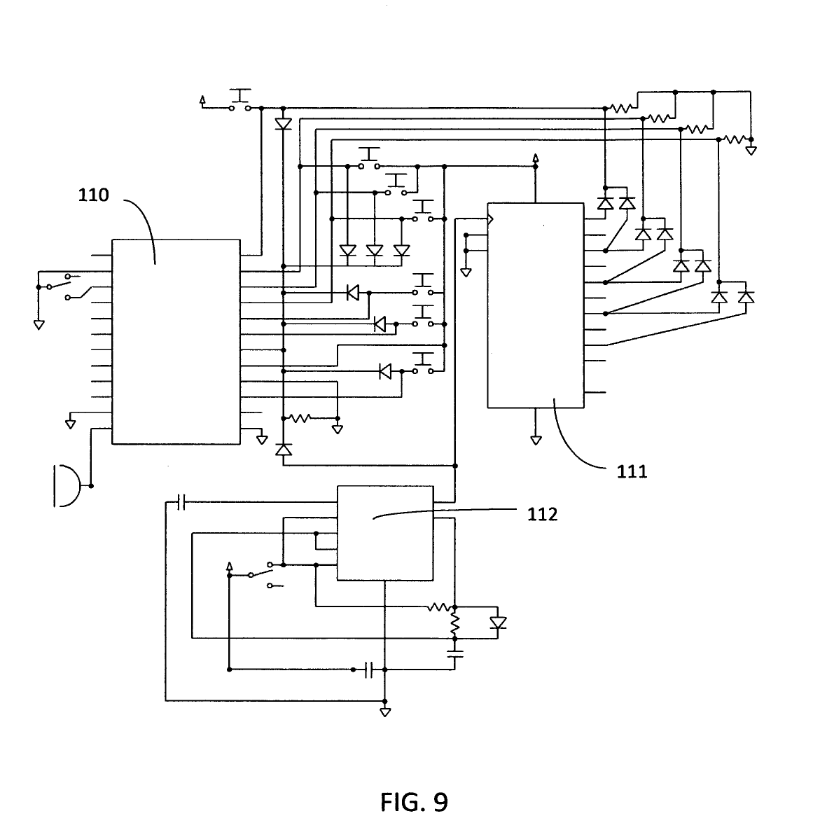

FIG. 9 describes the schematic diagram for one embodiment of the transmitter located within the remote control device 6. Standard electronic symbols are used to describe the component functions. Component 110 is a radio frequency transmitter/encoder microchip. Component 111 is a decoded Johnson ring counter microchip. Component 112 is a microchip timer used to drive the ring counter. This remote has programmed logic which enables sequenced activation of four vibration motor circuits while allowing independent operation of all vibration motor circuits by the user.

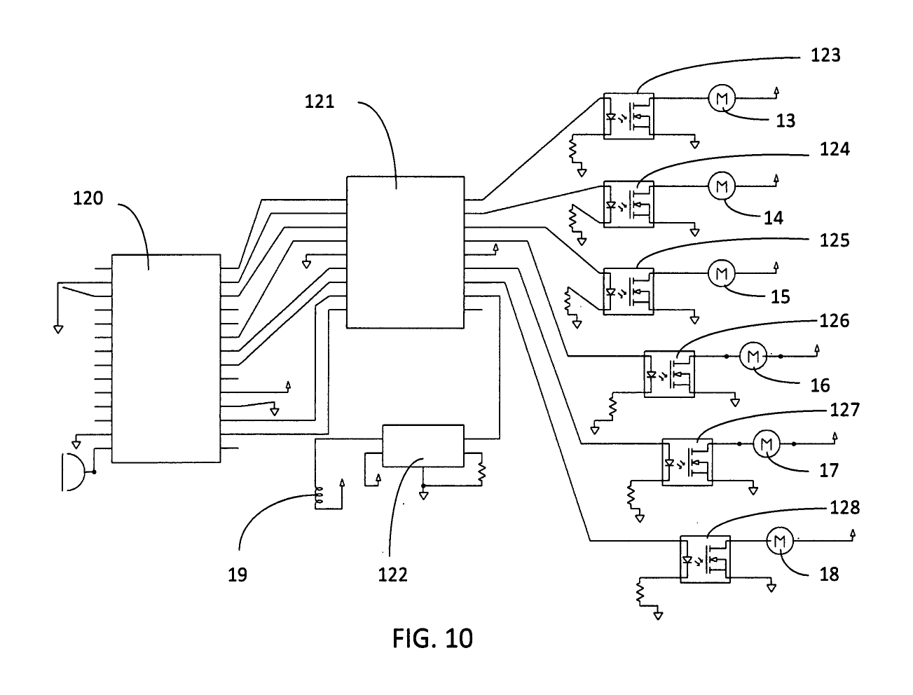

A detailed electric circuit diagram for a functional embodiment of the electric circuits 4 and 5 is shown in FIG. 10. Again, symbols indicated in the circuit diagram are traditional electrical symbols for describing various electronic components. Component 120 is a radio frequency receiver/decoder microchip. Component 121 is a momentary to latching toggle switch microchip. Component 122 is a constant current driver microchip. Components 123-128 are optically coupled, low loss semiconductor relay microchips which serve to turn the vibration motors on and off in response to commands sent by the remote. Output vibration motors 13-18 are indicated in the diagram and Heater 19 is also shown. This circuit is designed for use with batteries having 5-9 VDC input.

The embodiments of the invention described are only examples of a functioning wireless remote controlled foot massaging system. Other variations include; alternate methods for wireless remote operation of the electronics including RF, infrared, use of cell phones and personal computing devices and other modern methods, the addition or arrangement of other massaging components, (such as; Transcutaneous electrical nerve stimulation TENS and electrical muscle stimulation EMS devices or combination TENS/EMS devices) and modifications to the electronics circuits and remote control circuit for various functional modes of operation of the massaging components, the inclusion of operational indicator lights to the remote control and or shoes, use of other battery types and battery charging systems and integration and concealment of the wireless remote controlled electronics and battery into different locations including cross straps soles as appropriate for different style shoes. The battery and main switch can easily be located in a cross strap or the sole of the shoe if the shoe did not have an appropriate tongue or upper shoe portion for concealing the battery. The system can therefore be integrated into all types of footwear, men's shoes and women's shoes and high heels, sneakers, boots, sandals and slippers.

What is claimed to be new and novel and we pray to be secured by letters patent in the United States is described within the claims section:

1. A new wireless remote controlled massaging footwear system comprising a hand held wireless remote controller and footwear having a right shoe and left shoe each comprising a compact power supply and enclosed electronics for receiving wireless remote controlled signals from said wireless remote controller to functionally operate electrically powered massaging devices positioned within said right shoe and said left shoe.

2. The new wireless remote controlled massaging footwear system in claim 1 wherein said wireless remote controlled signal is a rolling radio frequency (RF) signal whereby the operation of subsequent wireless remote controlled footwear systems in close proximity is not affected.

3. The new wireless remote controlled massaging footwear system in claim 1 wherein said electrically powered massaging devices comprise a combination of multiple structurally supported miniature vibration motors.

4. The new wireless remote controlled massaging footwear system in claim 1 wherein said footwear comprises athletic shoes and said enclosed electronics are concealed within sole and tongue of said athletic shoes.

5. The new wireless remote controlled massaging footwear system in claim 1 wherein said electrically powered massaging devices comprise a structurally supported LED array.

6. The new wireless remote controlled massaging footwear system in claim 1 wherein said electrically powered massaging devices comprise TENS/EMS electrodes

7. The new wireless remote controlled massaging footwear system in claim 1 wherein said electrically powered massaging devices comprised of thin photo etched and laminated heating elements.

8. The new wireless remote controlled massaging footwear system in claim 1 wherein said remote controller selectively or sequentially operates individual said massaging devices within said footwear.

9. The new wireless remote controlled massaging footwear system in claim 1 wherein a first user or caregiver operates said wireless remote controller to functionally operate said footwear worn by a second user or patient.

10. A new wireless remote controlled massaging footwear system comprising a hand held wireless remote controller and footwear having a right shoe and left shoe each comprising a compact power supply and enclosed electronics for receiving wireless remote controlled signals from said wireless remote controller to functionally operate electrically powered massaging devices positioned within said right shoe and said left shoe, and soles having massage device holders for repositioning said massaging devices within said footwear.

11. The new wireless remote controlled massaging footwear system in claim 10 wherein said wireless remote controlled signal is a rolling radio frequency (RF) signal whereby the operation of subsequent wireless remote controlled footwear systems in close proximity is not affected.

12. The new wireless remote controlled massaging footwear system in claim 10 wherein said electrically powered massaging devices comprise a combination of multiple structurally supported miniature vibration motors.

13. The new wireless remote controlled massaging footwear system in claim 10 wherein said footwear comprises athletic shoes and said enclosed electronics are concealed within sole and tongue of said athletic shoes.

14. The new wireless remote controlled massaging footwear system in claim 10 wherein said electrically powered massaging devices comprise a structurally supported LED array.

15. The new wireless remote controlled massaging footwear system in claim 10 wherein said electrically powered massaging devices comprise TENS/EMS electrodes

16. The new wireless remote controlled massaging footwear system in claim 10 wherein said electrically powered massaging devices comprised of thin photo etched and laminated heating elements.

17. The new wireless remote controlled massaging footwear system in claim 10 wherein said remote controller selectively or sequentially operates individual said massaging devices within said footwear.

18. The new wireless remote controlled massaging footwear system in claim 10 wherein a first user or caregiver operates said wireless remote controller to functionally operate said footwear worn by a second user or patient.

19. A new wireless remote controlled massaging footwear system comprising;
a) a hand held wireless remote controller,
b) footwear comprising a right shoe and left shoe having
i) power supply,
ii) electrically powered massaging devices,
ii) enclosed electronics for receiving wireless remote controlled signals from said wireless remote controller to functionally operate electrically powered massaging devices positioned within said right shoe and said left shoe,
c) sole inserts to position said massaging components
d) a power supply charger,
e) optional lanyard for said hand held wireless remote control
f) optional belt clip for said hand held wireless remote control
f) optional carry case for said footwear system.

20. The new wireless remote controlled massaging footwear system in claim 19 wherein a first user or hospice caregiver operates said wireless remote controller to functionally operate said footwear worn by a second user or patient and said hospice caregiver carries said remote controller using said optional lanyard or belt clip and transports said system using said optional carry case, and said sole inserts are replaced or cleaned between patient use.
